SEQUENCE LISTING (12 sequences)

SEQ ID NO: 1 - DNA, 20 bp, Artificial Sequence
  16S rDNA sense primer for determine 16S rDNA base sequence
agagtttgatcmtggctcag

SEQ ID NO: 2 - DNA, 19 bp, Artificial Sequence
  16S rDNA antisense primer for determine 16S rDNA base sequence
ggttaccttgttacgactt

SEQ ID NO: 3 - RNA, 1385 nt, Cellulosimicrobium sp. HY-13
ugcaagucgaacgaugaugcccagcuugcuggguggauuaguggcgaacgggugaguaacacgugaguaaccugcccuugacuucgggauaacuccgggaaaccggggcuaauaccggauaugagccguccucgcaugggggugguuggaaaguuuuucggucagggaugggcucgcggccuaucagcuuguugguggggugauggccuaccaaggcgacgacggguagccggccugagagggcgaccggccacacugggacugagacacggcccagacuccuacgggaggcagcaguggggaauauugcacaaugggcgcaagccugaugcagcgacgccgcgugagggaugaaggccuucggguuguaaaccucuuucagcagggaagaagcgcaagugacgguaccugcagaagaagcgccggcuaacuacgugccagcagccgcgguaauacguagggcgcaagcguuguccggaauuauugggcguaaagagcucguaggcggucugucgcgucuggugugaaaacucgaggcucaaccucgagcuugcaucggguacgggcagacuagagugcgguaggggagacuggaauuccugguguagcgguggaaugcgcagauaucaggaggaacaccgauggcgaaggcaggucucugggccgcaacugacgcugaggagcgaaagcauggggagcgaacaggauuagauacccugguaguccaugccguaaacguugggcacuagguguggggcucauuccacgaguuccgugccgcagcaaacgcauuaagugccccgccuggggaguacggccgcaaggcuaaaacucaaaggaauugacgggggcccgcacaagcggcggagcaugcggauuaauucgaugcaacgcgaagaaccuuaccaaggcuugacaugcacgggaagccaccagagaugguggucucuuuggacacucgugcacagguggugcaugguugucgucagcucgugucgugagauguuggguuaagucccgcaacgagcgcaacccucgucccauguugccagcggguuaugccggggacucaugggagacugccggggucaacucggaggaagguggggaugacgucaaaucaucaugccccuuaugucuugggcuucacgcaugcuacaauggccgguacaaagggcugcgauaccguaagguggagcgaaucccaaaaagccggucucaguucggauuggggucugcaacucgaccccaugaagucggagucgcuaguaaucgcagaucagcaacgcugcggugaauacguucccgggccuuguacacaccgcccgucaagucacgaaagucgguaacacccgaagcccauggcccaaccguucgcgggggga

SEQ ID NO: 4 - RNA, 1671 nt, Cellulosimicrobium sp. HY-13
augaccaggaccaucuggagacgaccucucacgacgacgcucgcgaccacggcgcucgucgccgcgaccguccucccggucgccacgaccgcgaccgccgccaccgagccgcucggcgacgccgccgcgaggcacggcaagaccgucggcuucgcgcucgaccccggccgccucucggagagcggcuaccgggcggucgccgaccgcgaguucucgcucgucgucggggagaacgcgaugaagugggacgcgaccgaacccgcccggggcucguucuccuggggggcagcggaccgggucgcgagcuacgcggccgcgcagggcgcggaccucuacggccacacgcucguguggcaccaacagcuccccggcuggguccagggccugacgggcacggagcugcgcaccgccaugacggaccacguccgcgcggucgccgggcacuucgccggcgacgucgaggcgugggacgucgucaacgaggcguucgaggacgacggcucccgccggcagagcgucuuccagcagcgccucggagacggcuacaucgaggacgcccuccgugccgcacgggcggccgacccggacgcggaccugugccucaacgacuacagcaccgacgggaucaacgcgaagagcacggcgaucuacgaccucgucgccgacuucaaggcccgcggugugccgaucgacugcgucggguuccaggcgcaccugaucgugggccaggucccgucgacccugacccaggaccucaggcgguucgccgaccucggcguggacgugcgcaucaccgagcucgacauccgcaugaacacucccgcugacgcgcagaagcuggcgcagcaggcgucggacuacgcgaaggucuuccaggccugccucgacgucgaccgcugcacgggcgucacgcuguggggcaucaccgacagguacucguggaucccgggcguguucccggggcagggcgccgcgcucgucugggacgacgcguacgcgcccaagcccgcguacgcggcgaucgccgagguccucggcgcacgggacgacggaccgggcggcgacgagcaggcgccgucggccccgaccggccugcgggucaccggcaccacgaccucgucgaucucgcucgcguggaacgccuccaccgacgacgucggcgucgcgggguaccgcguguuccgcgacgggacccaggucgccgaggucgccgcgacgucguucaccgacaccggccucaccgcgggcaccgcgcacgucuacgcggugcgcgcggucgacgcggcgggcaaccucucggccacguccggcaccgugaccggcgagaccgaggaggguggcggcgaacccaccgggaccugcaccgucgccuacacggcaagcuccuggaacacgggcuucaccggcucgauccggaucaccaacgacucgacgaccgcgcugcacggcuggacccugagguucgcguucccggacgggcagaccguccagcagggcuggucggcgcaguacgcgcagcagggcucgaccgucaccgucaccccggcgccguggaacaccacgcucggcgcgggcgcgagcguggacaucggcuucaacggcgcccacuccgggaucaacaccgagccgaccuccuucacgcucgacggugccgccugcgaggucgccuga

SEQ ID NO: 5 - protein, 556 aa, Cellulosimicrobium sp. HY-13
MTRTIWRRPLTTTLATTALVAATVLPVATTATAATEPLGDAAARHGKTVGFALDPGRLSESGYRAVADREFSLVVGENAMKWDATEPARGSFSWGAADRVASYAAAQGADLYGHTLVWHQQLPGWVQGLTGTELRTAMTDHVRAVAGHFAGDVEAWDVVNEAFEDDGSRRQSVFQQRLGDGYIEDALRAARAADPDADLCLNDYSTDGINAKSTAIYDLVADFKARGVPIDCVGFQAHLIVGQVPSTLTQDLRRFADLGVDVRITELDIRMNTPADAQKLAQQASDYAKVFQACLDVDRCTGVTLWGITDRYSWIPGVFPGQGAALVWDDAYAPKPAYAAIAEVLGARDDGPGGDEQAPSAPTGLRVTGTTTSSISLAWNASTDDVGVAGYRVFRDGTQVAEVAATSFTDTGLTAGTAHVYAVRAVDAAGNLSATSGTVTGETEEGGGEPTGTCTVAYTASSWNTGFTGSIRITNDSTTALHGWTLRFAFPDGQTVQQGWSAQYAQQGSTVTVTPAPWNTTLGAGASVDIGFNGAHSGINTEPTSFTLDGAACEVA

SEQ ID NO: 6 - DNA, 18 bp, Artificial Sequence
  xy1K1 sense primer for pcr
tgggacgtcstcaacgag

SEQ ID NO: 7 - DNA, 18 bp, Artificial Sequence
  xy1K1 antisense primer for pcr
gatgtcgagctcsgtgat

SEQ ID NO: 8 - DNA, 22 bp, Artificial Sequence
  delta Fn3 sense primer for pcr
catatggccaccgagccgctcg

SEQ ID NO: 9 - DNA, 25 bp, Artificial Sequence
  delta Fn3 antisense primer for pcr
aagctttcaggacctcggcgatcgc

SEQ ID NO: 10 - protein, 293 aa, Cellulosimicrobium sp. HY-13
LGDAAARHGKTVGFALDPGRLSESGYRAVADREFSLVVGENAMKWDATEPARGSFSWGAADRVASYAAAQGADLYGHTLVWHQQLPGWVQGLTGTELRTAMTDHVRAVAGHFAGDVEAWDVVNEAFEDDGSRRQSVFQQRLGDGYIEDALRAARAADPDADLCLNDYSTDGINAKSTAIYDLVADFKARGVPIDCVGFQAHLIVGQVPSTLTQDLRRFADLGVDVRITELDIRMNTPADAQKLAQQASDYAKVFQACLDVDRCTGVTLWGITDRYSWIPGVFPGQGAALVWDD

SEQ ID NO: 11 - protein, 72 aa, Cellulosimicrobium sp. HY-13
PSAPTGLRVTGTTTSSISLAWNASTDDVGVAGYRVFRDGTQVAEVAATSFTDTGLTAGTAHVYAVRAVDAAG

SEQ ID NO: 12 - protein, 100 aa, Cellulosimicrobium sp. HY-13
CTVAYTASSWNTGFTGSIRITNDSTTALHGWTLRFAFPDGQTVQQGWSAQYAQQGSTVTVTPAPWNTTLGAGASVDIGFNGAHSGINTEPTSFTLDGAAC

(19) United States
(12) Patent Application Publication
(10) Pub. No.: US 2012/0309074 A1
(43) Pub. Date: Dec. 6, 2012

(54) NOVEL XYLANASE PRODUCED FROM CELLULOSIMICROBIUM FUNKEI HY-13

(76) Inventors: Ho-Yong Park, Daejeon (KR); Kwang-Hee Son, Daejeon (KR); Do Young Kim, Daejeon (KR); Tae-Sook Jeong, Daejeon (KR); Sung Uk Kim, Daejeon (KR); Dong-Ha Shin, Daejeon (KR)

(21) Appl. No.: 13/577,811

(22) PCT Filed: Mar. 18, 2010
(86) PCT No.: PCT/KR10/01693

§ 371 Date: Aug. 8, 2012

(30) Foreign Application Priority Data
Feb. 9, 2010 (KR) 10-2010-0011970

(51) Int. Cl. C12N 9/24; C12N 1/19; C12N 5/10; C12N 1/21

(52) U.S. Cl. 435/200; 435/252.33; 435/254.2; 435/325; 435/348

(57) ABSTRACT

There are provided a novel xylanase and a use of the same. In detail, there are provided a xylanase separated from a Cellulosimicrobium funkei HY-13 strain, a Fibronectin Type 3 domain of the xylanase, and a use thereof. Since determining that the xylanase having substrate specificity degrades xylan at neutral and basic pH with high efficiency and the Fn3 domain does an important role with respect to the substrate specificity, the xylanase according to the present invention may be added to various vegetable feed materials or be efficiently used to improve degradation ability of cellulosic biomass.

### BACKGROUND OF THE INVENTION

1. Field of the Invention

The present invention relates to a microorganism producing novel xylanase,

2. Description of the Related Art

A cell wall of plants, which is a maximum storage for fixed carbon existing in nature, includes three important compounds such as cellulose that is insoluble β-1,4-glucan cellulose, hemicellulose that is a non-cellulose polysaccharide composed of glucan, mannan, and xylan, and lignin with a polyphenolic structure. Hemicellulose binds tightly a cellulose fascicle and strongly maintains biomass to be structurized, which becomes recalcitrance against hydrolyzing lignocellulosic biomass to use. To perfectly degrade and saccharify xylan composing such hemicelluloses, generally, three enzymes such as endo-β-xylanase, exo-β-xylanase, and β-xylosidase should be reacted together with one another, which are commonly called as xylanase enzymes. Xylan includes 30%of sugar in hemicellulose pasture includes 20% of sugar in hemicellulose of feed legumes. Since hemicellulose of feed legumes includes a glycoconjugates more complicated than that of hemicellulose of pasture, there is required an enzyme resolving celluloses in order to hydrolyze hemicellulose of feed legumes with a more complicated structure. When adding xylanase to feed legumes, a hemicellulose membrane covering a grain is degraded, thereby increasing the utilizability of nutrients in grains and also improving a state of digesting grains in intestines of domestic animals. Particularly, in case of bioethanol recently receiving attention as green energy, technologies is transited from a first generation bioethanol whose raw material is maize starch to the second generation bioethanol whose raw material is fibers, thereby improving pretreatment of cellulosic biomass using expert enzymes. Accordingly, xylanase has a great value as an enzyme for being added to teed and also has high utilizability as enzymes for producing bio-energy.

Most of microorganism used to produce xylanase for feed, which has been reported, there are fungi. Among them, strains of genus Trichoderma sp. are generally used. Fungi belonging to the genus Trichoderma sp. slowly grow, which lengthens a culture time thereof and becomes difficulties in genetic usage and variation. On the other hand, when using microorganism such as bacteria, proliferation and genetic transition thereof are easy and industrial utilizability is high. For industrial usage, it is urgently required to select bacteria capable of producing xylanase. Also, xylanase produced by a strain belonging to the genus Trichoderma sp. is activated at most with an acid condition around pH 5.0. However, since, though physiological conditions of digestive organs of pigs or chickens are different to depending on a part, a pH condition of subsequent organs of small intestines, in which xylanase reacts, is around 6.5, there is a limitation of activity performance when applying to a corresponding field on xylanase derived from fungi, though with high experimental enzymatic activity. Accordingly, xylanase is required as an enzyme for an addition to feed, whose optimal enzyme activity corresponds to a pH condition in intestines of domestic animals.

Invertebrates including insects are well thriving groups on earth and present various feeding habits and high biological variety. Recently, considering such living properties of invertebrates, there are increased researches for using symbiotic microorganism of the invertebrates as beneficial bio-resources. Particularly, there are vigorously performed researches on rumen microorganisms, closely related to the growth of invertebrates. For example, in intestines of termites, microorganism related to degrading wood that is food for termites compose a community and are involved in digestion and nutrients. Strains producing high efficient protease are separated from diadem spiders and industrially used. Also, there are reported researches on biology of rumen microorganisms of various invertebrates composed of Lepidoptera and Coleoptera using molecular biological technology. Also, to increase activity of enzymes, there are applied various microbiological, molecular-biological technologies. Particularly, since a structure of protein provides original technology most important to activate enzymes, there has been continued an effort to develop high efficient enzymes via various genetic manipulations and transformations in structure of protein.

Accordingly, the present inventors selected strains producing high efficient xylanase, which produce novel xylanase XylK1, from intestines of a large number of invertebrates such as earthworms whose food was vegetable remains in soil. It was determined that xylanase separated from the producing strains highly were activated at neutral and alkaline pH and degraded sugar substrate including xylan and produced xylooligosaccharides of X4 to X7 using xylotriose X3 and xylotetraose X4 as substrates. Also, the present invention was completed by determining that it was possible to make good use of the xylanase as a material improving feed efficiency and an enzyme hydrolyzing biomass by determining that the xylanase was composed of a fibronectin type 3 domain (Fn3 domain) and the Fn3 domain took a great role in determining activity of enzymes and a binding capacity thereof with substrate.

### SUMMARY OF THE INVENTION

To solve the problems as described above, the present invention provides novel xylanase and a method for using the same.

To achieve the goal as described above, according to an aspect of the present invention, there is provided xylanase including the following properties (a) through (h):

(a) a molecular mass of about 42 kDa on SDS-PAGE;

(b) a pI value of 4.49;

(c) maximum activity at pH 5 to 9;

(d) maximum activity at a temperature of 55° C.;

(e) a mesophilic enzyme;

(f) endo-β-1,4-xylanase and

(g) a GH10 (glycoside hydrolase family 10 domain, an Fn3 (Fibronetic Type 3) domain, and a CBM2 (carbohydrate-binding module2) domain.

The present invention provides a polynucleotide encoding the xylanase.

The present invention provides a recombinant expression vector to which the polynucleotide is operatively linked.

The present invention provides a transformant formed by introducing the recombinant expression vector to a host cell.

According to another aspect of the present invention, there is provided a method of producing a xylanase, the method including the steps:

(1) yielding a crude enzyme solution by culturing and centrifuging the transformant;

and

(2) purifying a xylanase from the crude enzyme solution yielded in Step (1).

The present invention provides a xylan degradation agent including one of the xylanases, the xylanase produced according to the method, and the transformant.

The present invention provides a composition for producing xylan in food, the composition including one of the xylanases, the xylanase produced according to the method, and the transformant.

The present invention provides a composition for paper manufacture, the composition including one of the xylanases, the xylanase produced according to the method, and the transformant.

The present invention provides feed additives including one of the xylanases, the xylanase produced according to the method, and the transformant, as an active component.

The present invention provides feed grain with increased xylan glycemic index, the feed grain including the feed additives.

According to still another aspect of the present invention, there is provided a method of manufacturing feed, the method including the step: adding one of the xylanases, the xylanase produced according to the method, and the transformant to a feed material for animal.

According to yet another aspect of the present invention, there is provided a method of degrading xylan, the method including the step: adding one of the xylanases, the xylanase produced according to the method, and the transformant to one of cellulosic biomass and xylan solution.

The present invention provides a use of one of the xylanases, the xylanase produced according to the method, and the transformant to manufacture a composition for producing xylan in food.

The present invention provides a use of one of the xylanases, the xylanase produced according to the method, and the transformant to manufacture a composition for paper manufacture.

The present invention provides a use of one of the xylanases, the xylanase produced according to the method, and the transformant to manufacture a composition for manufacturing feed additives.

The present invention provides a GH10 (glycoside hydrolase family 10) xylanase separated from Cellulosimicrobium funkei HY-13 highly activates at neutral and alkaline pH and degrades sugar substrate including xylan produces xylooligosaccharides of X2 to X7 using xylotriose X3 and xylotetraose X4 as substrate. Accordingly, the xylanase according to the present invention may be usefully used as an agent for improving feed efficiency and an enzyme for hydrolyzing biomass.

### BRIEF DESCRIPTION OF THE DRAWINGS

The application of the preferred embodiments of the present invention is best understood with reference to the accompanying drawings, wherein:

FIG. 1 illustrates a result of investigating homologe between a polypeptide sequence of a novel xylanase separated from the present invention and polypeptide sequences of GH10 xylanases registered in NCBI, the GH10 xylanase including: Cellulosimicrobium sp. A strain HY-13 (Csp) xylanase (FJ859907); a Cellulomonas fimi (Cfi) xylanase (AAA56792); Streptomyces ambofaciens (Sam) xylanase (CAJ88420); Acidothermus cellulolyticus 11B (Ace) xylanase (ABK51955); and Thermobifida alba (Tal) xylanase (CAB02654).

In this case, a black box indicates the same amino acid and a gray box indicates pseudo-amino acids, respectively;

a guessed signal peptide is presented as a black bar;

a highly conserved amino acid residue taking a great role in an enzyme reaction is presented as *; and

GH10 (glycoside hydrolase ID), Fn3 (fibronectin type 3), and CBM2 (carbohydrate-binding module 2) are presented as an unbroken line, a long-dotted line, and a dotted line, respectively.

### DESCRIPTION OF THE PREFERRED EMBODIMENTS

Features and advantages of the present invention will be more clearly understood by the following detailed description of the present preferred embodiments by reference to the accompanying drawings. It is first noted that terms or words used herein should be construed as meanings or concepts corresponding with the technical sprit of the present invention, based on the principle that the inventor can appropriately define the concepts of the terms to best describe his own invention. Also, it should be understood that detailed descriptions of well-known functions and structures related to the present invention will be omitted so as not to unnecessarily obscure the important point of the present invention.

Hereinafter, the present invention is described in detail.

The present invention provides a xylanase including the following properties:

(a) a molecular mass of about 42 kDa on SDS-PAGE;

(b) pI value of 4.49;

(c) maximum activity at pH 5 to 9;

(d) maximum activity at a temperature of 55° C.;

(e) mesophilic enzyme;

(f) endo-β-1,4-xylanase; and

(g) a GH10 (glycoside hydrolase family 10) domain, an Fn3 (Fibronetic Type 3) domain, and a CBM2 (carbohydrate-binding module2) domain.

The xylanase according to the present invention also produces xylooligosaccharide using xylotriose and xylotetraose as substrates.

The Fn3 domain may include an amino acid sequence represented by SEQ. No. 11 but not limited thereto. Any Fn3 domain known to those skilled in the art may be within the scope of the present invention.

In exemplary embodiments of the present invention, a strain with excellent ability of degrading xylan from an intestinal extract of invertebrates was identified and a novel xylanase was separated from the strain by using conserved sequences.

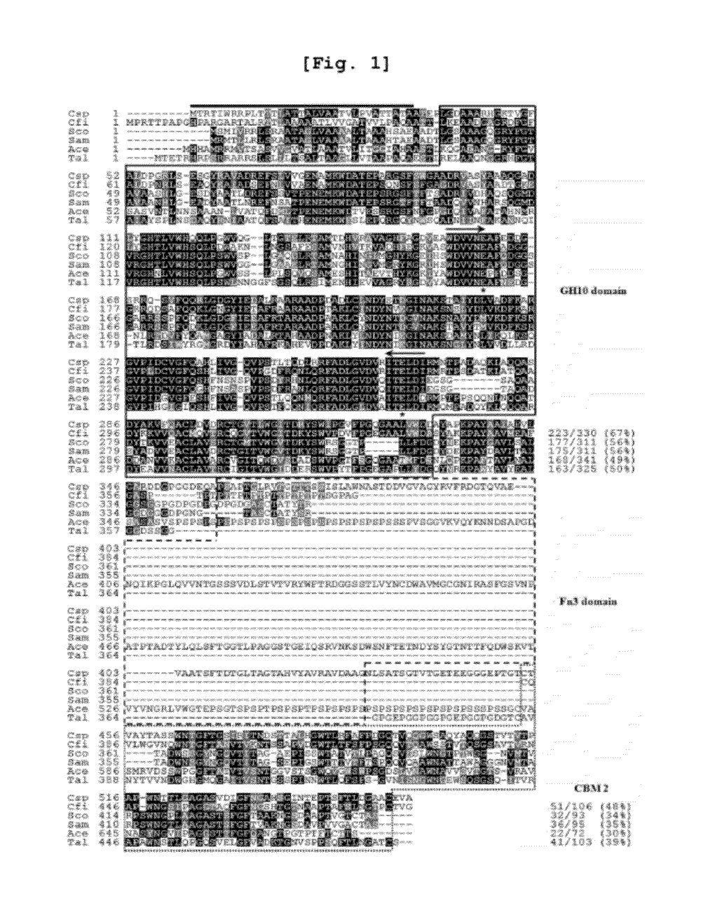

Also, in exemplary embodiments of the present invention, a primer was manufactured from an area where a sequence and aromatic characteristics of a GH10 xylanase generally reported were conserved, a polynucleotide sequence encoding the GH10 xylanase from gDNA of the strains was cloned to a protein expression vector and expressed in E. coli, a recombinant xylanase enzyme (rXylk1) was purified, and properties thereof was investigated. As result thereof, it was presented that the xylanase according to the present invention included molecular mass of about 42.0 kDa. Also, comparing protein sequences of other GH10 sylanases obtained from an NCBI database with a protein sequence induced from the an Xylk1 polynucleotide, antithetically to protein sequences of other GH10 xylanases, the Xylk1 was confirmed as a single unit xylanase including an N-terminal enzyme activity GH10 domain (sequence number 10, Leu38 to Asp330), an Fn3 domain (sequence number 11, Pro359 to Gly430), and a C-terminal CBM2 domain (sequence number 12, Cys454 to Cys553) (refer to FIG. 1.). Though, in conventional, Flavobacterium johnsoniae UW101 reported a unit xylanase (Genbank approach number ABQ06877) including, an N-terminal Fn3 domain and a C-terminal GH10 domain via genome researches, proteinic properties thereof were not disclosed. Except for this, there was nothing reported with respect to a GH10 xylanase including Fn3. Also, an enzymatic GH10 domain of Xylk1 presented sequential homologe of 67% with a Cellulomonas fimi xylanase AAA 56792, among GH10 enzymes available in the NCBI database, and a CBM2 domain of C-terminal presented sequential homologe of 64% with Cellulomonas fimi GH6 cellulase AAC36898. An Fn3 domain of Xylk1 presented highest sequential homologe of 70% with Acidothermus cellulolyticus 11B GH48 enzyme ABK52390 hydrolyzing cellulose. Also, the highest enzymatic activity was presented at pH 6.0 and enzymatic activity of 80% or more was maintained at pH 5.0 to 9.0. Also, the highest activity was presented at a temperature of 50 to 60° C., and more particularly, at a temperature of 55° C. Also, the activity of rXylk1 was decreased to 40% by Hg2+ and decreased to 25% by Ca2+, Cu2+, and Ba2+, was stable with respect to Mn2+ and Co2+, and was increased by Fe2+. Also, the enzymatic activity of rXylk1 was decreased by EDTA but relatively less afflicted by sulfhydryl reagents such as sodium azide, iodoacetamide, and N-ethylmalemide. Also, the xylanase according to the present invention was perfectly suppressed by 5 mM N-bromosuccinimide and the enzymatic activity thereof was significantly increased by adding one of Tween 80 and Triton X-100. Also, checking an influence of an Fn3 domain on enzymatic activity by using the recombinant xylanase rXylk1 and mutant rXylk1ΔFn3 whose Fn3 domain was truncated, the Fn3 domain truncation of rXylk1 did not induce a significant change from associative sociability with respect to oat spelt xylan. However, since it was determined that rXylk1ΔFn3 was bound with the oat spelt xylan but not bound to Avicel, it was known that an Fn3 domain took an important role in an enzyme-substrate association (refer to Table 1). Celluloses composing grains and wood were surrounded with xylan required to be degraded to separate the celluloses. From the result above, a xylanase including an Fn3 domain, whose accessibility with substrate was improved, efficiently catalyzed the hydrolysis of xylan in a process of treating grains and biomass. Also, since the xylanase according to the present invention presented a high ability of degrading birchwood xylan while with an Fn3 domain (refer to Table 2), it was determined that the xylanase including an Fn3 domain according to the present invention not only was better bonded to substrate but also more efficiently degraded sastrate associated in practice.

Accordingly, as results of sequential analysis and activity analysis, it was so determined that the xylanase produced from identified strains according to the present invention was novel from other conventional xylariases.

Also, the xylanase according to the present invention may include any one of the following amino acids:

a) an amino acid sequence represented by SEQ. No. 5;

b) an amino acid sequence with homologe of 70% or more with the amino acid sequence represented by SEQ. No. 5;

c) an amino acid sequence encoded by a base sequence represented by SEQ. No. 4;

d) an amino acid sequence composed by substituting, deleting, insetting and/or adding one or more amino acids in, from, into and/or to the amino acid sequence represented by SEQ. No. 5 and composing protein with the same function as that of protein including the amino acid sequence represented by SEQ. No. 5; and

e) an amino acid sequence encoded by, a DNA hybridized with a DNA including the base sequence represented by SEQ. No. 4 under a stringent condition, the amino acid of protein with the same function as that of the protein including the amino acid sequence represented by SEQ. No. 5,

but not limited thereto.

The stringent condition of e) is determined when washing after the hybridization. One of stringent condition is washing at room temperature with 6±SSC, 0.5% SDS for 15 minutes, washing at a temperature of 45° C. with 2±SSC, 0.5% SDS for 30 minutes, and washing at a temperature of 50° C. with 0.2±SSC, 0.5% SDS for 30 minutes and repeated twice. More preferably, a temperature higher than the above is used. As another of the stringent condition, other parts of the stringent condition are identically performed and washing of the last two times of 30 minutes is performed at a temperature of 60° C. with 0.2±SSC, 0.5% SDS, As still another of the stringent condition, the last two times of washing are performed at a temperature of 65° C. with 0.1±SSC, 0.1% SDS. It is obvious to those skilled in the art to set up such limitations to obtain the required stringent condition.

The xylanase according to the present invention may activates maximally at pH 5 to 9, and more particularly, at p171 6 and may activates at a temperature of 50 to 60° C. and more particularly, at a temperature of 55° C., but is not limited thereto.

The xylanase according to the present invention may be derived from a Celluosimicrobium funkei HY-13 strain deposited as Deposit No. 11302BP but not limited thereto.

In the exemplary embodiments of the present invention, a bacterial colony produced by streaking intestinal extract of invertebrates on a medium for separating bacteria containing 0.5% of birchwood xylan was cultured in a culture solution including 0.5% of birchwood xylan at a temperature of 25° C. for two days, and strains with excellent ability of degrading xylan were selected by using a cultural supernatant as a crude enzyme solution, and microorganism producing a xylanase were separated. The separated strains were an ectosymbiosis group and gram positive bacteria. As a result of investigating homologe with respect to 16S rDNA base sequence, the separated strains presented high homologe of 99.8° C. or more with a Cellulosimicrobium funkei ATCC BAA-886 strain, there identifying the present strain as Cellulosimicrobium funkei and designating the same by Cellulosimicrobium funkei HY-13. The Cellulosimicrobium funkei HY-13 strain was deposited in Korean Collection for Type Cultures (KCTC) in Korea Research Institute of Bioscience and Biotechnology, international deposit institution, on Mar. 12, 2008 (refer to Deposit No. KCTC 11302BP).

Also, the present invention provides a polynucleotide encoding the xylanase. The polynucleotide encoding the xylanase may include one of the following base sequences:

a) a base sequence represented by SEQ. No. 4;

b) a base sequence having 95% of homologe with the base sequence represented by SEQ. No. 4;

c) a base sequence encoding an amino acid sequence represented by SEQ. No. 5;

d) a base sequence encoding an amino acid sequence composed by substituting, deleting, inserting and/or adding one or more amino acids in from, into and/or to the amino acid sequence represented by SEQ. No. 5 and composing protein with the same function as that of protein including the amino acid sequence represented by SEQ. No. 5; and

e) a base sequence of a DNA hybridized with a DNA including the base sequence represented by SEQ. No. 4 under a stringent condition, the base sequence of protein with the same function as that of protein including the amino acid sequence represented by SEQ. No. 5,

but not limited thereto.

Also, the present invention provides a recombinant expression vector to which the polynucleotide is operatively linked.

Since the present invention discloses base sequences of a novel DNA encoding xyalanase separated from a Cellulosimicrobium funkei HY-13 strain, a recombinant vector including the DNA may be manufactured using a general method well known to those skilled in the art. The recombinant vector according to the present invention may be a commercialized vector but not limited thereto. Also, it is permissible that those skilled in the art manufacture and use a proper recombinant vector.

The present invention also provides a transformant formed by introducing the recombinant vector to a host cell.

A host cell available in the present invention is not limited but may be one selected from the group consisting of a prokaryotic cell including E. coli, yeast, an animal cell, and a eukaryotic cell including an entomic cell. More preferably, the host cell is a colon bacillus but not limited thereto.

The present invention also provides a method of manufacturing a xylanase, the method including the steps:

1) yielding a crude enzyme solution by culturing and centrifuging the transformant; and

2) purifying a xylanase from the crude enzyme solution yielded in the step 1).

The step 2) may include the following steps:

1) introducing water soluble protein to be precipitated by adding a precipitant to a supernatant yielded by centrifuging a culture solution of the transformant;

2) yielding the crude enzyme solution by removing and dialyzing insoluble precipitates from the precipitates of 1); and

3) purifying the crude enzyme solution of 2) using column chromatography,

but not limited thereto.

In the above, the medium may be one of the Cellulosimicrobium funkei HY-13 strain and one, suitable for the transformant of the present invention selected from media generally used and well-known to those skilled in the art.

In the above, the precipitant of the step 1) may be one selected from the group consisting of ammonium sulfate, acetone, isopropanol, methanol, ethanol, and polyethylene glycol. The precipitation may be replaced by ultrafiltration using a film with various pore sizes and concentration.

In the above, the column chromatography may be performed using a tiller selected from the group consisting of silica gel, Sephadex RP-18, polyamide, Toyopearl, and XAD resin. The column chromatography may be performed several times selecting a suitable filler.

Also, the present invention provides a xylan degradation agent including one of the xylanases, the xylanase produced according to the method, and the transformant.

The xylan degradation agent may be one of the strain and the xylanase produced from the transformant and may also be the transformant.

Also, the present invention provides a composition for producing, xylan in food, the composition including one of the xylanases, the xylanase produced according to the method, and the transformant.

Also, the present invention provides a composition for paper manufacture, the composition including one of the xylanases, the xylanase produced according to the method, and the transformant.

The composition according to the present invention may include the xylanase according to the present invention and a component identical or similar thereto and may contain the xylanase according to the present invention with 1 to 90% but not limited thereto.

Since the xylanase according to the present invention, different from conventional xylanase, derived from fungi, presenting low hydrolysis activity under neutral and alkaline conditions, presents high activity under neutral and alkaline conditions (pH 5 to 9) and has xylose substitutive-activity enabling production of long xylooligosaccharide from X3 and X4, it is possible to use the xylanase according to the present invention as a xylanase highly activating under wide pH condition.

Also, the present invention provides feed additives including one of the xylanases, the xylanase produced according to the method, and the transformant, as an active component.

In exemplary embodiments, the xylanase according to the present invention presented highest activity at pH 6.0 but the activity thereof was maintained more than 80% within pH 5.0 to 9.0. Considering that xylanases derived from fungi are acid xylanases and have lower activity at neutral pH, since having high activity within neutral pH and alkaline pH, the xylanase according to the present invention is considered to have high applicability as enzyme supplements added to feed.

Also, in exemplary embodiments of the present invention, the enzyme had highest cleaving activity with respect to PNP-cellobioside, higher than that with other xylanases known as the same substrate (Haga K M et. al., 1991; Kim K Y et al., 2009. Proc. Biochem.: 1055-1059). Also, the xylanase according to the present invention was determined to have high degradation ability with respect to birchwood xylan, beech wood, xylan, oat spelt xylan, and PNP(p-nitrophenyl)-cellobioside but not to degrade soluble starch, Avicel, and carboxyl methylcellulose, thereby determining the xylanase according to the present invention to be real Enod-β-1,4-xylanase, inactive with cellulase. Additionally, the xylanase according to the present invention was determined to have xylose substitution activity capable of cleaving PNP-xylopyranoside.

From the result above, it was determined that the xylanase according to the present invention had particularity and efficiently degraded xylan. Considering that feed grain generally used for animals substantially contain xylan, the xylanase according to the present invention is efficient for animal feed. Checking the result as described above, the xylanase according to the present invention was determined to be suitable for feed additives to increase degradation of xylan in feed grain.

Accordingly, the xylanase produced according to the method according to the present invention may be usefully applied as feed additives saccharification of xylan.

The feed additives according to the present invention may be added to feed for non-ruminant animals such as pigs and chickens, whose efficiency of using starch or protein of grain, in cell walls, due to the absence of enzymes capable of degrading cell walls, and may saccharify xylan primary component of cell walls, thereby improving the value of the feed.

The xylanase according to the present invention, which is an active component of feed additives, may consist 0.01 to 10 parts by weight of feed, more particularly, consist 0.05 to 5 parts by weight of the feed, and most particularly, consist 0.1 parts by weight of the feed.

Also, the feed additives may further contain a carrier allowable to non-ruminant animals. In the present invention, the feed additives may be provided alone or adding a well-known carrier and a stabilizer. When necessary, all sorts of nutrients such as vitamin. amino acids, and minerals, antioxidant, and other additives may be added, whose shape may be convenient therefor, such as powder, granule, pellet, and suspension. When supplying the feed additives according to the present invention, the feed additives may be supplied alone or mixed with feed to non-ruminant animals.

Also, the present invention provides feed grains with increased saccharification of xylan including the feed additives as an active component.

Currently, a xylanase may be commercially used in the fields of food, feed, and technology (Bedford and Morgana, World's Poultry Science Journal 52: 61-68, 1996). In the food market such as production of fruits and vegetables, brewing and manufacturing alcoholic beverages, breadmaking and confectionaries, the xylanase is used to soften materials, to improve refinement efficiency, to reduce viscosity, and to improve quality by increasing efficiency of extraction and filtration. In the feed market, the xylanase is used to reduce nonstarch carbohydrates, to improve viscosity in intestines, and increase a digestion-absorption rate of protein and starch in feed of pigs, poultries, and ruminant animals (Kuhad and Singh, Crit. Rev. Biotechnol. 13, 151-172, 1993). In addition, technologically, the xylanase is used to biologically whiten paper in a paper manufacture process, to reduce consumption of chlorines, to reduce energy by shortening a mechanical paper manufacture process, to generate deinking efficiency, to separate starch from gluten, and to manufacture recyclable fuel such as bioethanol and chemical raw material.

Therefore, the novel xylanase according to the present invention may be usefully applied to manufacture paper and recycle waste paper, to improve the quality of feed additives and food, and to be used in xylanase degradation that is industrially used, which is well-known to those skilled in the art. The compositions may be formulated and manufactured as a raw material by methods well-known to those skilled in the art.

Also, the present invention provides a method of manufacturing feed, the method including the step: adding one of the xylanase, the xyalanse produced according to the method, and the transformant to a feed material for animals.

In the method, an added amount of one of the strain, the transformant, and a xylanase produced by one of the transformant and the strain may be adjusted by those skilled in the art.

Also, the present invention provides a method of degrading xylan, the method including the step: adding one of the xylanases, the xylanase produced according to the method, and the transformant to one of cellulosic biomass and xylan solution.

The xylan degradation method may be applied to a process of producing recyclable fuel or a chemical raw material but not limited thereto. In the xylan degradation method, an addition amount of adding one of the strain, the transformant, and the xylanase produced by one of the transformant and the strain may be adjusted by those skilled in the art.

Also, the present invention provides a use of one of the xylanases, the xylanase produced according to the method, and the transformant to manufacture a composition for producing xylan in food.

The xylan degradation agent according to the present invention may be used to manufacture a composition for producing xylan in food, since it is possible not only to use the xylanase produced by one of the strain and the transformant but also to use one of the strain and the transformant as the xylan degradation agent.

Also, the present invention provides a use of the xylanase, the xylanase produced according to the method, and the transformant to manufacture a composition for paper manufacture.

Additionally, the present invention provides a use of the xylanase, the xylanase produced according to the method, and the transformant to manufacture feed additives.

When manufacture one of the composition for paper manufacture and feed additives by using the composition according to the present invention, the composition may include the xylanase according to the present invention and one the same as the xylanase or similar thereto and may include the xylanase according to the present invention 1 to 90% of the entire composition but not limited thereto.

Since the xylanase according to the present invention, different from conventional xylanases, derived from fungi, presenting low hydrolysis activity under neutral and alkaline conditions, presents high activity under neutral and alkaline conditions (pH 5 to 9) and has xylose substitutive-activity enabling production of long xylooligosaccharide from X3 and X4, it is possible to use the xylanase according to the present invention as a xylanase highly activating under wide pH condition.

Hereinafter, the present invention will be described in detail with reference to experimental examples and formulation examples. However, the following experimental examples and formulation examples are provided only for illustrative purpose of the present invention, and the present invention is not limited by the following experimental examples and formulation examples.

### Embodiment 1. Separate and Select Strain Producing Xylanase from Invertebrates

The present inventors collected earthworms (Eisenia fetida) used in investigating microorganism with a xylanase producing activity in nearby Daejon, brought the earthworms alive to the laboratory, and classified the earthworms to use. To separate bacteria producing a xylanase, the surface of the earthworms was cleaned using ethanol and rinsed three times using distilled water. The cleaned sample was dissected, and internal organs thereof were separated, putted into a PBS buffer solution (0.8% of NaCl, 0.02% of KCl, 0.144% of Na2HPO4, and 0.024% of KH2PO4), and ground. An extract thereof was diluted by stages, was streaked on a solid medium to which 0.5% of birchwood xylan had been added, cultured at a temperature of 25° C. for three days, and after that, strains forming a clear zone around a colony where microorganism had grown were selected primarily via a Congo-red dying method (Theater R M & Wood P J, Appl. Environ. Microbiol. 43: 777-780, 1982). The strains selected as described above were inoculated to 3 ml of a limiting medium containing 0.5% of birchwood xylan, (K2HPO4 7 g/L, KH2PO4 2 g/L, (NII4)2SO4 1 g/L, MgSO4.7H2O 1.1 g/L, and enzyme extracts 0.6 g/L) and were cultured in a shaking incubator at a temperature of 25° C. for 48 hours. A supernatant thereof were recovered by centrifugation and the activity of the xylanase was measured. Among them, strains with an excellent xylanase activity were finally selected. In this case, the enzyme activity was performed using DNS (Dinitrosalicylic acid) quantitative method (Miller G L, Anal. Chem., 55: 952-959, 1959). In detail, 350 μl of a substrate solution (1% of birchwood xylan) and 50 μl of 0.5 M phosphoric acid buffer solution (pH 6.0) were added to 100 μl of an enzyme solution and were reacted therewith at a temperature of 55° C. for 10 minutes. After that, 750 μl of DNS (3,5-Dinitrosalicylic acid) solution were added thereto, left alone at a temperature of 100° C. fix 5 minutes, and measured at 540 nm of absorbance. One unit of enzymes was determined to be an enzyme amount discharging 1 μmol of reducing sugar for one minutes.

### Embodiment 2. Identify Separated Strain

The strains separated from intestines of earthworms and selected in Embodiment 1 were identified.

The separated strains are ectosymbiosis, exists on an intestinal mucous membrane, and gram positive bacteria.

Also, to determine 165 rDNA base sequence of microorganism, a genome DNA of the strains were separated and were PCR reacted with the composition as follows. In detail, to 1 μl, of a genome DNA (50 to 100 ng/μl), 2 μl often times a Tag DNA polymerase buffer solution (MgCl2 added), 2 μl of 2.5 mM dNTPs, 1 μl of a forward primer (27F: 5′-agagtttgatcmtggctcag-3′, SEQ. No. 1) and a reverse primer (1492R: 5′-gghaccttgttacgactt-3′, SEQ. No. 2) of 10 pmol, respectively, and 1 to 2 units of a Tag DNA polymerase (Promega, USA) were added, and finally, distilled water was added thereto to prepare 50 μl of a reaction solution. In this case, a pair of the primers were manufactured to amplify 1373 bp of a nucleotide, corresponding to 16S rDNA part of eukaryotic bacteria. PCR is performed denaturing at a temperature of 94° C. for 5 minutes, denaturing at a temperature of 944° C. for 30 seconds, annealing at a temperature of 50° C. for 30 seconds, extending at a temperature of 72° C. for 3 minutes, repeated 30 times, and finally, extending at a temperature of 72° C. for 7 minutes and maintaining at a temperature 4° C.

As a result of determining the 165 rDNA base sequence as SEQ. No. 3 and investigating homologe, there was shown high homologe of 99.8% or more with Cellulosimicrobium funkei ATCC BAA-886 strains, thereby the present strains were identified to be Cellulosimicrobium funkei and were designated as Cellulosimicrobium funkei HY-13. The Cellulosimicrobium funkei HY-13 strains were deposited in Korean Collection for Type Cultures (KCTC) in Korea Research institute of Bioscience and Biotechnology, international deposit institution, on Mar. 12, 2008 (refer to Deposit No. KCTC 11302BP).

### Embodiment 3, Clone and Purify Xylanase

<3-1> Cloning of Xylanase

The present inventors amplified and cloned a polynucleotide sequence (SEQ. NO. 4) encoding xylanase protein (SEQ. No. 5) by using primers manufactured based on a sequence of an area (WDVVNE and ITELDI) conserved from GH10 (glycoside hydrolase in family 10) xyalanase in a genome DNA of the strains selected in Embodiment 2. In detail, the genome DNA was separated from the strains, and PCR was performed with respect to a xylanase DNA, with the genome DNA as a template, by using 10× buffer solution (MgCl2), 2.5 mM dNTPs, 5×GG-rich buffer solution, a FastStart Taq DNA polymerase (Roche), and a pair of primers including a sense primer (5′-TGG GAC GTC STE AAC GAG-3′), represented by SEQ. No. 6, and an antisense primer (5′-GAT GTC GAG CTC SGT GAT-3′), represented by SEQ. No. 7. In this case, the PCR is performed under a condition as follows: denaturing at a temperature of 95° C. for 5 minutes, denaturing at a temperature of 95° C. for 30 seconds, annealing at a temperature of 50° C. for 30 seconds, extending at a temperature of 72° C. for 40 seconds, repeated 35 times, and finally, extending at a temperature of 72° C. for 7 minutes. Genome walking and nested-PCR were performed on a PCR product of 342 bp of a xylanase, yielded via the PCR, by using a DNA Walking SpeedUp premix kit (Seegene, Korea), thereby yielding a PCR product with respect to the entire xylK1 gene. The PCR product of the entire xylK1 gene and pET-28a(+) vector (Novagen, USA) were cleaved using Nde I and Hind II limiting enzymes and purified. About 100 ng of the purified vector and the PCR product were used, respectively, and one unit of ligase (TaKaRa Company) was added thereto and reacted at a temperature of 16° C. for 16 hours. After ligation reaction, the vector were transformed to BL21 (Novagen), selected from a plate containing kanamycin, and cleaved to be a suitable limiting enzyme, thereby acquiring plasmid with a preferable DNA slice. A clone was determined finally by DNA sequencing. The manufactured expression vector was designated as ‘pET-xylK1’.

Also, “pET-XylK1ΔFn3” expression vector formed by deleting a Fibronectin Type 3 domain and a CBM 2 (carbohydrate-binding module 2) domain from the entire xylK1 gene was manufactured by cloning using the same method as described above except fix using a pair of primers including a sense primer (5′-CAT GCC ACC GAG CCG CTC G-3′), represented by SEQ. No. 8, and an antisense primer (5′-AAG CTT TCA GGA CCT COG CGA TCG C-3″), represented by SEQ. No. 9.

<3-2> Purify Xylanase

One of pET-xylK1 and pET-XylK1ΔFn3 expression vectors was overexpressed in E. coli, and one of a recombinant XylK1(rXylK1) and a recombinant XylK1ΔFn3(rXylK1ΔFn3) was separated. In detail, E. coli formed by transforming the respective expression vectors were inoculated to a liquid LB medium and cultured, being shaken, at a temperature of 37° C. When OD600 values of respective coliform culture solutions amounted to 0.4 to 0.5, 1.0 mM of IPTG was added thereto and the solutions were further cultured, being shaken, at a temperature of 30° C. for 5 hours. The culture solutions were centrifuged and cells were ground using a sonicator to be observed. As a result of observation, it was determined that rXylK1 was overexpressed from active inclusion bodies and rXylK1ΔFn3 was overexpressed from inactive inclusion bodies. Accordingly, the present inventors ground cells of the E. coli overexpressing rXylK1 and solubilized the inclusion bodies thereof. One of the solubilized rXylK1 cell-ground material and rXylK1ΔFn3 cell-ground material were refolded and purified using HisTrap HP (GE Healthcare, Sweden) (5-ml) column, and high-performance liquid chromatography (LC) system (Amersham Pharmacia Biotech, Sweden) was performed thereon according to a manual of the Company thereof. There was determined Electrophoretic homogeneity of one of rXylK1 and rXylK1ΔFn3 proteins purified by performing Gel permeation chromatography using a HiLoad 26/60 Superdex 200 prep-grade (Amersham Biosciences Sweden), well-known those skilled in the art.

One of the rXylK1 and rXylK1ΔFn3 proteins purified above was quantitated using Bradford reagent (Bio-Rad, USA.), freeze-dried, and kept at a temperature of −20° C.

### Embodiment 4, Properties of Xylanase

<44> Sequencing

The present inventors compared protein sequences of other GH10 xylanases obtained from the NCBI database with protein sequences induced from XylK1 polynucleotide according to the present invention. By contrast to other GH10 the XylK1 according to the present invention was determined as a single unit xylanase including an N-terminal enzyme activity GH10 domain (SEQ. No. 10, leu38 to Asp330), an Fn3 domain (SEQ. No. 11, Pro359 to Gly430), and a C-terminal CBM2 domain (SEQ. No. 12, Cys454 to Cys553). There was reported an uncharacterized modular xylanase (GenBank Accession No. ABQ06877) including an N-terminal Fn3 domain and a C-terminal GH10 domain via genome research in Flavobacterium johnsoniae UW101. Except for this, there was not reported a GH10 xylanase including Fn3.

As shown in FIG. 1, an enzymatic GH10 domain of XylK1 presented highest sequence homologe of 67% with a Cellulomonas fimi xylanase (AAA 56792) among GH10 enzymes available in the NCBI database. However, CRM 2 of the enzyme presented homologe of 64% with Cellulomonas fimi GH6 cellulase (AAC36898). The Fn3 domain of XylK1 presented highest sequence homologe of 70% with Acidothermus cellulolyticus 11B GH48 enzyme (ABK52390) degrading cellulose. Two conserved residues of Glu161 (acid/base catalyst) and Glu266 (catalyst eukaryotic body) were predicted in the active site of premature XylK1.

<4-2> Molecular Mass Analysis

SDS-PAGE was performed on the xylanase purely separated in Embodiment 3-2 in a gel of 12%, and it was determined that the xylanases had about 42 KDa and about 34 KDa. As a result of performing MALDI-TOF MS (Matrix-assisted laser desorption ionization time-of-flight mass spectrometry) analysis using MALDI-TOF mass spectrometer (Broker Daltonics, Germany) in Korea Basic Science Institute (Daejeon, Korea), it was measured that His-tagged, purified rXylK1 had a molecular mass of 45, 169 Da, protein smaller than intact rXylK1. The above result, inducing from a property of being tightly bind to a His tag column and the molecular mass of protein becoming smaller, was determined as occurring because the rXylK1 was expressed in E. coli and cleaving of proteins thereof occurred in a C-terminal area due to certain protein hydrolysis enzymes derived from host cells. It was assumed that Val439-Thr440 site was processed in a hinge area between an Fn3 domain and the C-terminal CMB2 of the premature. XylK1 and became intact rXylK1 based on a measured molecular mass of truncated rXylK1, 45,169 Da. The deduced molecular mass(45,179 Da) of rXylK1 with the Val439 residue at the Cterminal extremity was very close to the molecular mass(45,169 Da) of the enzyme calculated by MALDI-TOF MS analysis

<4-3> Biochemical Properties

To investigate an optimum reaction condition of rXylK1, there were checked the effect of a reaction pH, a temperature, metal ions, a reagent, and a surfactant. In detail, an optimum pH of enzymatic activity was measured using 50 mM of a sodium citrate buffer solution with pH 3.5 to 5.5, 50 mM of a phosphate buffer solution with pH 5.5 to 7.5, 50 mM of Tris-HCl buffer solution with pH 7.5 to 9.0, and 50 mM of glycine-NaOH buffer solution with pH 9.0 to 10.5. An optimum temperature of enzymatic activity was measured from 30 to 70° C. at intervals of 5° C. An effect of metal ions on enzymatic activity was measured under reaction conditions including 1 mM of one of Hg2+, Ca2+, Cu2+, Ba2+, Mn2+, Co2+, and Fe2+, respectively. An effect of a reagent was measured under reaction conditions including 5 mM of EDTA, sodium azide, iodoacetamide, and N-ethylmaleimdie, respectively. An effect of a surfactant was measured under a reaction condition including one of 0.5% of Tween 80 and Triton X-100.

As a result thereof, rXylK1 presented highest activity at pH 6.0 and presented 80% or more of activity within pH 5.0 to 9.0. Considering that a xylanase derived from fungi is an acid xylanase and activity thereof is low at a neutral pH, since rXylK1 highly activates also at a neutral pH, rXylK1 may be well used as enzyme supplements.

Also, rXylK1 presented maximum activity at a temperature of 55° C.

Also, the activity of rXylK1 was reduced by 40% with Hg2+ and reduced by 25% with Ca2+, Cu2+, and Ba2+. A xylanase derived from Streptomyces sp, strain S9 (Kulkarni N A et al, 1999, FEMS microbial. Rev, 23: 411 to 456), and Aeromonas caviae ME-1 (Liu C. J T et al., 2003. J. Biosci. Bioeng. 95: 95-101) were negatively affected by Mn2+ and Co2+. However, the xylanase according to the present invention was stable with respect to the ions. Previously, there was reported that enzymatic activity was restrained by Fe2+ (Hasa K M et al., 1991. Agric. Biol. Chem. 55: 19591967; Oh H W et al., 2008. Antonie van Leeuwenhoek 93: 437442), enzymatic activity of rXylK1 was increased by about 1.4 times with Fe2+. Also, when 5 mM of EDTA was precultured for 10 minutes, original activity of the enzyme was lost by 68%. Opposite thereto, rXylK1 was relatively less affected by sulthydryl reagents such as sodium azide, iodoacetamide, and N-ethylmaleimide. As presented by Streptomyces lividans) (Roberge M R et al., 1999. Protein Eng. 12: 251257) and T-6 (Geobacillus stearothermophilus T-6) (Zolotnitsky G U et al., 2004. Proc. Natl. Acad. Sci. USA 101: 11275-11280) GH10 xylanase, perfect inhibition of rXylK1 due to 5 mM of N-broinosuccinimide well explains that a Trp residue in an area of highly conversed GH10 enzymes are importantly included in an enzyme-substrate interaction. It is expected that three residues Trp118, Trp306, and Trp314 of incomplete XylK1 do an important role in binding of an enzyme with a catalyst and a substrate. The enzymatic activity of His-tagged rXylK1 was noticeably increased by about 1.8 times when adding one of Tween 80 and Triton X-100 with a concentration of 0.5%. In addition, stimulation of the activity of the His-tagged rXylK1 was not noted when adding a surfactant without preculturing, a composition having the same enzymatic reaction for 10 minutes This implies that the activity of nonionic-surfactant-inducible His-tagged rXylK1 occurred while the recombinant enzyme directly, mutually acts with one of Tween 80 and Triton X-100 molecules, which causes a variation of enzyme-substrate interaction.

<4-4> Sabstrate Specificity

To check how the enzymatic activity of the xylanase according to the present invention with respect to a carbohydrate polymer varies with the existence of an Fn3 domain, a binding capacity of one of rXylK1 and rXylK1ΔFn3 with a carbohydrate polymer was measured. Above all, to check a binding capacity of one of rXylK1 and rXylK1ΔFn3 with an insoluble sugar substrate, a binding capacity with one of Avicel and insoluble oat spelt xylan was measured using a well-known method (Cazernier A E al., 1999. Appl. Environ. Microbiol, 65: 4099 to 4107). In this case, a binding capacity of one of rXylK1 and rXylK1ΔFn3 with birchwood xylan was measured and used as a comparison group. Also, to check whether the existence of the Fn3 domain influences not only substrate-specific binding but also hydrolysis of actually bound xylan, degradation ability of one of rXylK1 and rXylK1ΔFn3 with the birchwood xylan was measured using the method of Embodiment 1. in addition, degradation abilities of rXylK1 according to the present invention with various xylans and a sugar substrate shown in Table 3 were checked using the method of Embodiment 1. In this case, as a comparison group, 0.5 ml of a standard analysis mixture including 0.05 ml of an enzyme solution manufactured by diluting one of 1.0% of birch wood xylan and 5 mM of a PNP (p-nitropheriyl) sugar derivative with 50 mM of sodium phosphate buffer solution (pH 6.0) was enzymatically reacted at a temperature of 55° C. for 10 minutes and compared therewith. One unit of xylanase activity with respect to one of xylan and PNP-sugar derivative was defined as an amount of enzymes required to produce 1 μmol of one of a reducing sugar and PNP for one minute under a standard analysis condition. Enzymatic hydrolysis of 10 mg of birchwood xylan (Sigma Co.), 1 mg of xylooligosaccharide (Megazyme International Ireland, Ireland), and 1 mg of cellooligosaccharide (Seikagaku Biobuisness Co., Japan) were performed by reacting using 2 μg of purified rXylK1 for 3 to 6 hours while stability of the enzymes were maintained, under a condition including 0.1 ml of 50 mM sodium phosphate buffer solution (pH 6.0) at a temperature of 37° C.

As a result thereof, as shown in Table I, it was checked that there is no noticeable difference of binding affinity with a carbohydrate polymer between rXylK1ΔFn3 and rXylK1, which presented that a C-terminal truncation of rXylK1 did not induce a noticeable variance in the binding affinity between the enzyme and the carbohydrate polymer. Also, it was checked that rXylK1ΔFn3 was able to be bound with insoluble oat spelt xylan and to facilitate hydrolysis of a xylan polymer as rXylK1 but was not bound with Avicel, different from rXylK1. This presents that an Fn3 domain does an important role in the enzyme-substrate binding.

TABLE 1

Activity of unit xylanase
after binding
(total IU)a

| Substrate | rXylK1 | rXylK1ΔFn3 |
|---|---|---|
| Comparison group | 0.50 | 0.50 |
| Avicel | ≦0.01 | 0.49 ± 0.02 |
| Insoluble oat spelt | 0.05 ± 0.01 | ≦0.01 |
| xylan | | |

athe activity of a unit xylanase, analyzed using birchwood xylan

Also, as shown in Table 2, comparing with rXylK1ΔFn3, the activity of rXylK1 was higher by 5.3 times. From this, it was checked that the xylanase having an Fn3 domain was not only substrate-specifically bound but also hydrolyzed a xylan polymer actually bound therewith.

TABLE 2

| | Specific activity of xylanase, IU/mg | | |
|---|---|---|---|
| | | | Ratio between |
| Substrate | rXylK1 (A) | rXylK1ΔFn3 (B) | A and B |
| Birch wood | 143.0 | 27.0 | 5.3:1 |
| xylan | | | |

Also, as shown in Table 3, among evaluated xylan materials, oat spelt xylan was most effectively hydrolyzed by rXylK1. Also, there was not observed activity of rXylK1 with respect to other such as soluble starch, Avicel, and carboxyl methylcellulose. Enzymatic activity of rXylK1 with respect to the PNP-cellobioside was higher than activity of the enzyme with respect to oat spelt xylan (193 IU/mg) by about 1.7 times. Accordingly, it was checked that the xylanase according to the present invention had no degradation ability with respect to glucose-based starch. Also, cleaving activity of the rXylK1 according to the present invention with respect to PNP-cellobioside is about 48 IU/mg, higher than the cleaving activity with respect to other xylanases known as the same substrate (10 IU/mg) (Haga K M at al., 1991: Kim D Y et al., 2009. Proc. Biochem. 44: 1055 to 1059). The result indicates that rXylK1 is true endo-β-1,4-xylanase inactive with cellulose. In addition, it was checked that rXylK1 had about 7.5% of maximum hydrolysis activity of the enzyme with respect to xylose substitution activity capable of cleaving PNP-xylopyranoside (oat spelt xylan).

TABLE 3

| Substrate | Relative activity (%)^{a} |
|---|---|
| Birchwood xylan | 74.1 ± 2.8 |
| Beach wood xylan | 85.8 ± 3.5 |
| Oat spelt xylan | 100.0 |
| Soluble starch | Nothing detected |
| Avicel | Nothing detected |
| Carboxyl methylcellulose | Nothing detected |
| PNP-cellulobioside | 171.7 ± 4.9 |
| PNP-glucopyranoside | ≦0.5 |
| PNP-xylopyranoside | 7.5 ± 0.6 |

^{a}Relative activity obtained by an experiment repeated three times

<4-5> Properties of Xylan Degradation Product

The reaction mixture in Embodiment 4-4 was heated at a temperature of 100° C. for 5 minutes and an enzymatic reaction was at standstill, and a hydrolysis product was measured performing LC-MS using a mobile phase of elution solution A (0.05% of pomalus acid/sterile water) and elution solution B (0.05% of pomalus acid/sterile water:acetonitrile/methanol 6:4) according to a well-known method (Kim D Y et 2009).

As a result thereof, as shown in Table 4, when adding xylotriose (X3) and xylotetraose (X4) as a substrate of hydrolysis reaction with respect to rXylK1 enzyme, there was observed a xylose substitution reaction. Though it was known that X2 and X3 are primary products, when enzymatically hydrolyzing X3 at a temperature of 37° C. for three hours, there were produced xylooligosaccharides X4 to X7. Similar to this, hydrolysis of X4 by rXylK1 produced a mixture including 42.3% of long xilooligosaccharides X5 to X8. This indicates that the xylooligomer was produced by rXylK1-catalyst xylose, substitution reaction. However, X1 was not detected as a hydrolysis product of one of X2, X3, and X4. An ability of rXylK1 to catalyze synthesis of long xylooligosaccharide from one of the X3 and X4 is very particular in an aspect that a microbial xylanase generally produces short xylooligosaccharide such as one of X2 and X3 from the same substrate (Brennan Y et al,, 2004. 70: 3609-3617; Oh H W et al., 2008). Also, rXylK1 degraded birchwood xylan to 65.1% of X2, 29.5% of X3, and 5.4% of X4 at a temperature of 37° C. for six hours.

TABLE 4

| | Composition (%)^{a} of product formed by | | | | | | |
|---|---|---|---|---|---|---|---|
| | hydrolysis reaction | | | | | | |
| Substrate | X2 | X3 | X4 | X5 | X6 | X7 | X8 |
| X2 | 100.0 | | | | | | |
| X3 | 27.8 | 45.4 | 16.8 | 5.6 | 3.9 | 0.5 | |
| X4 | 12.8 | 26.3 | 18.6 | 18.1 | 14.5 | 8.4 | 1.3 |
| Birchwood | 65.1 | 29.5 | 5.4 | | | | |
| xylan | | | | | | | |

^{a}LC area percentage

1-18. (canceled)

19. A xylanase comprising one of the following amino acid sequences:
a) an amino acid sequence represented by SEQ. No. 5;
b) an amino acid sequence with homologe of 70% or more with the amino acid sequence represented by SEQ. No. 5;
c) an amino acid sequence encoded by a base sequence represented by SEQ. No. 4;
d) an amino acid sequence composed by substituting, deleting, inserting and/or adding one or more amino acids in, from, into and/or to the amino acid sequence represented by SEQ. No. 5 and composing protein with the same function as that of protein comprising the amino acid sequence represented by SEQ. No. 5; or
e) an amino acid sequence encoded by a DNA hybridized with a DNA comprising the base sequence represented by SEQ. No. 4 under a stringent condition, the amino acid of protein with the same function as that of the protein comprising the amino acid sequence represented by SEQ. No. 5.

20. The xylanase of claim 19, wherein the xylanase is derived from a Cellulosimicrobium funkei HY-13 strain deposited as Deposit No. KCTC 11302BP.

21. The xylanase of claim 19, wherein the xylanase is enclosed by a polynucleotide, the polynucleotide comprising one of the following base sequences:
a) a base sequence represented by SEQ. No. 4;
b) a base sequence having 95% of homologe with the base sequence represented by SEQ. No. 4;
c) a base sequence encoding an amino acid sequence represented by SEQ. No. 5;
d) a base sequence encoding an amino acid sequence composed by substituting, deleting, inserting anclior adding one or more amino acids in, from, into and/or to the amino acid sequence represented by SEQ. No. 5 and composing protein with the same function as that of protein comprising the amino acid sequence represented by SEQ. No. 5; and
e) a base sequence of a DNA hybridized with a DNA comprising the base sequence represented by SEQ. No. 4 under a stringent condition, the base sequence of protein with the same function as that of protein comprising the amino acid sequence represented by SEQ. No. 5.

22. A transformant introducing a recombinant expression vector, to which the polynucleotide encoding the xyhmse of claim 19 is operatively linked, to a host cell.

23. The transformant of claim 22, wherein the host cell is one of a prokaiyotic cell comprising E. coli and a eukaryotic cell comprising yeast, an animal cell, and an insect cell.

24. A method of producing a xylanase, the method comprising the steps:
1) yielding a crude enzyme solution by culturing and centrifuging the transformant of claims 22; and
2) purifying the xylanase from the crude enzyme solution yielded in the step 1).

25. The method of claim 24, wherein the step 2) comprises:
1) introducing water soluble protein to be precipitated by adding a precipitant to a supernatant yielded by centrifuging a culture solution of the transformant of claim 24;
2) yielding the crude enzyme solution by removing and dialyzing insoluble precipitates from the precipitates of the step 1); and
3) purifying the crude enzyme solution of the step 2).

26. The xylanase of claim 19, comprising the following properties:
(a) a molecular weight of about 42 kDa on SDS-PAGE;
(b) a pI value of 4,49;
(c) maximum activity at pH 5 to 9;
(d) maximum activity at a temperature of 55° C.;
(e) a mesophilic enzyme;
(f) endo-β-1,4-xylanase; and
(g) a GH10 (glycoside hydrolase family 10) domain, an FnS (Fibronetic Type 3) domain, and a CBM2 (carbohydrate-binding module2) domain.

27. The xylanase of claim 26, further comprising the property of producing a xylooligosaccharide using xylotriose and xylotetraose as substrates.
